(19) United States
(12) Patent Application Publication
(10) Pub. No.: US 2012/0244075 A1
(43) Pub. Date: Sep. 27, 2012

(54) 5 NM Nickel-NTA-Gold Nanoparticles

(75) Inventors: Wenqiu Liu, Dix Hills, NY (US); James F. Hainfeld, Shoreham, NY (US)

(73) Assignee: Nanoprobes, Inc., Yaphank, NY (US)

(21) Appl. No.: 13/072,591

(22) Filed: Mar. 25, 2011

Related U.S. Application Data
(60) Provisional application No. 61/467,350, filed on Mar. 24, 2011.

(51) Int. Cl. A61K 49/00; A61P 35/00; B32B 5/16; A61K 9/16; B82Y 5/00

(52) U.S. Cl. 424/9.1; 428/403; 424/491; 977/773; 977/915; 977/927

(57) ABSTRACT

The present disclosure relates to the product, process, and use of 5 nm Nickel-Nitrilotriacetic acid (Ni-NTA) gold nanoparticles. Applications include diagnostic tests, imaging, therapies, detection technologies, gold conjugation to other molecules, and novel material constructs.

### CROSS-REFERENCE

This application claims priority to U.S. Provisional Application No. 61/467,350, filed Mar. 24, 2011, the contents of which are incorporated in their entirety by reference herein.

### BACKGROUND

Nanotechnology holds great promise for the development of effective diagnostic and therapeutic methods for diseases such as cancer, atherosclerosis, and stroke, as well as uses in basic biomedical and material science research and applications.

A 1.8 nm Nickel-NTA-gold (Ni-NTA-gold) nanoparticle has previously been described (e.g. See Hainfeld et al., J Struct Biol. 1999 September;127(2):185-98). It has become apparent after several years of use that this particle has a number of shortcomings, including: a) it is very small and difficult to detect even by standard electron microscopy, b) its extinction coefficient is low, making it difficult to directly detect in assays by eye or a reader, c) it delivers a low amount of gold per labeled molecule, d) there is background or non-specific binding to some other non-His-tagged proteins or materials, and e) its x-ray absorption is low.

### SUMMARY OF THE INVENTION

The present disclosure provides compositions comprising a plurality of gold nanoparticles bound to at least one multidentate ligand, such as a tetradentate ligand chelated to Nickel (II), wherein at least about 90% of the plurality of gold nanoparticles have an effective diameter about 5 nm±25% and methods of preparing same.

In another aspect, the present disclosure also provides compositions comprising a plurality of gold nanoparticle conjugates, comprising a plurality of gold nanoparticles bound to at least one multidentate ligand such as a tetradentate ligand chelated to Nickel (II), wherein at least about 90% of the plurality of gold nanoparticles have an effective diameter about 5 nm±25% and methods of preparing same.

While the constructions of a specific metal nanoparticle (gold) are given, the procedures are applicable to functionalizing other metal nanoparticles made from the metal group scandium, titanium, vanadium, chromium, manganese, iron, cobalt, nickel, copper, zinc, yttrium, zirconium, niobium, molybdenum, ruthenium, rhodium, palladium, silver, cadmium, mercury, hafnium, tantalum, tungsten, rhenium, osmium, iridium, platinum, gold, gadolinium, aluminum, gallium, indium, tin, thallium, lead, bismuth, magnesium, calcium, strontium, barium, lithium, sodium, potassium, boron, silicon, phosphorus, germanium, arsenic, antimony, and combinations thereof.

In some embodiments, the methods for functionalization with chelating groups are applicable to other shapes of metal nanoparticles including nanorods, nanoshells (metal with non-metal core), metal nanoparticle with hollow core, cubic, triangular, tetrahedral, and other shaped nanoparticles. In other embodiments, the methods are applicable to nanoparticles of various sizes, about 2 to about 1,000 nm, and even macro particles about 1 to about 50 μm.

In some embodiments, the methods are also applicable to functionalization with other chelating groups including multiple carboxyl groups, iminodiacetic acid, tris(carboxymethyl) ethylenediamine, ethylenediaminetetraacetic acid, diethylene triamine pentaacetic acid, and DOTA (1,4,7,10-tetraazacyclododecane-1,4,7,10-tetraacetic acid). Although a specific chelated metal (nickel) is described in detail, other metals may be used in the method including scandium, titanium, vanadium, chromium, manganese, iron, cobalt, copper, zinc, yttrium, zirconium, niobium, molybdenum, ruthenium, rhodium, palladium, silver, cadmium, mercury, hafnium, tantalum, tungsten, rhenium, osmium, iridium, platinum, gold, gadolinium, aluminum, gallium, indium, tin, thallium, lead, bismuth, magnesium, calcium, strontium, barium, lithium, sodium, potassium, boron, silicon, phosphorus, germanium, arsenic, antimony, and combinations thereof. The uses described may be optimized by optimal combinations of the nanoparticle components. For example, for MRI imaging, gadolinium is a preferred metal.

In some embodiments, provided herein are compositions applicable to high resolution molecular labeling for electron and light microscopy, providing a signal for detection, providing a method for specific binding to a target molecule, and providing a method to construct nanoparticle conjugates with macromolecules and other materials. In one embodiment, the 5 nm Ni-NTA gold nanoparticles are used in diagnostic tests including lateral flow, ELISA, dot blots, chips, and other formats. In yet further embodiments, the functionalized nanoparticles are used in vivo to provide imaging of various targets by various technologies such as x-ray CT or planar x-rays, MRI, PET, and SPECT. In further embodiments, the 5 nm Ni-NTA gold nanoparticles are used for therapies including enhancement of radiotherapy and drug delivery.

### DESCRIPTION OF DRAWINGS

FIG. 1 illustrates schematic of 5 nm Ni-NTA-gold nanoparticle binding to His-tagged protein.

FIGS. 2A and 2B show TEM micrograph of about 5 nm Ni-NTA Gold (2A: 5.11±0.84 nm, scale bar=20 nm; 2B: 4.77±0.84 nm; scale bar=20 nm).

FIG. 3 is a picture of a Dot blot showing 0.5 ng detection of a 6x-His-tagged protein using Ni-NTA-5 nm gold nanoparticles. Spots left to right have targets of His-tagged ATF-1 protein at a loading of 100 ng, 50 ng, 10 ng, 5 ng, 1 ng, and 0.5 ng per spot. A second row of control proteins (all spots identical), E. Coli extract (1 μL of 2.03 mg/ml total protein, 2.03 μg per spot), was spotted directly below the test target ATF-1 protein spots.

FIG. 4 shows Electron micrograph of T7 bacteriophage with 6x-His-tags expressed on the coat capsomeres. The Ni-NTA-5 nm gold nanoparticles were then mixed and the virus purified by gel filtration chromatography. Bar=20 nm.

FIG. 5 shows Electron micrograph of T7 phage with 6x-His-tag fusions binding Ni-NTA-5 nm gold nanoparticles to form a supramolecular structure.

FIG. 6 shows a picture of Ni-NTA-5 nm gold nanoparticles detecting 6x-His-tagged proteins on western blot.

### DETAILED DESCRIPTION

It is to be understood that the foregoing general description and the following detailed description are exemplary and explanatory only and are not restrictive of any subject matter claimed. In this application, the use of the singular includes the plural unless specifically stated otherwise. It must be noted that, as used in the specification and the appended claims, the singular forms “a,” “an” and “the” include plural referents unless the context clearly dictates otherwise. In this application, the use of “or” means “and/or” unless stated otherwise. Furthermore, use of the term “including” as well as other forms, such as “include”, “includes,” and “included,” are not limiting.

Unless defined otherwise, all technical and scientific terms used herein have the same meaning as is commonly understood by one of skill in the art to which the claimed subject matter belongs.

All publications and patents mentioned herein are incorporated herein by reference in their entirety for the purpose of describing and disclosing, for example, the methodologies that are described in the publications, which might be used in connection with the presently described inventions. The publications discussed herein are provided solely for their disclosure prior to the filing date of the present application. Nothing herein is to be construed as an admission that the inventors described herein are not entitled to antedate such disclosure by virtue of prior invention or for any other reason.

The term “acceptable” with respect to a formulation, composition or ingredient, as used herein, means having no persistent detrimental effect on the general health of the subject being treated.

The term “bound,” as used herein refers to one or more associations, interactions, or bonds that are covalent or non-covalent (including ionic bonds, hydrogen bonds, and van der Waals interactions).

The term “carrier,” as used herein, refers to relatively nontoxic chemical compounds or agents that facilitate the transport of metal nanoparticles into vasculature, tissues, or cells.

The terms “co-administration” or the like, as used herein, are meant to encompass administration of the selected therapeutic agents to a single patient, and are intended to include treatment regimens in which the agents are administered by the same or different route of administration or at the same or different time.

As used herein, “EC₅₀” refers to a dosage, concentration or amount of metal nanoparticles that elicits 50% of a maximal effect that is induced, provoked, or potentiated by the metal nanoparticles.

The term “effective amount,” refers to the amount of metal nanoparticles that is required to obtain a therapeutic or diagnostic effect in combination with a therapeutically effective dose of infrared irradiation. A “therapeutically effective amount,” as used herein, refers to an amount of metal nanoparticles sufficient to allow detection of a target when the metal nanoparticles are provided to the therapeutic target and the therapeutic target is exposed to a therapeutically effective dose of infrared irradiation or sufficient to relieve to some extent one or more of the pathological indicia associated with the therapeutic target. The result can be reduction and/or alleviation of the signs, symptoms, or causes of a disease, or any other desired alteration of a biological system. For example, an “effective amount” for therapeutic uses is the amount of metal nanoparticles as disclosed herein required to provide a clinically significant decrease in disease symptoms or other pathological indicia without undue adverse side effects. It is understood that “an effective amount” or “a therapeutically effective amount” can vary from subject to subject, due to variation in therapeutic target size, shape, depth, composition, as well as systemic factors such as circulation, metabolism, age, weight, general condition of the subject, the severity of the therapeutic target-associated condition being treated, and the judgment of the prescribing physician.

As used herein, the term “infrared” refers to any wavelength between about 700 to about 1100 nm.

The term “metal nanoparticle,” as used herein refers to a nanoparticle that has a core mass which is at least about 50%, about 60%, about 70%, about 80% or about 90% metallic by weight. A metal nanoparticle includes nanoparticles that are composed essentially of metal atoms.

The term “nanoparticle,” as used herein, refers to an object of any shape that can be contained in a spherical volume having a diameter of about 1000 nm or less (i.e., has an effective diameter of about 1000 nm or less) unless stated otherwise.

The term “non-target,” as used herein, refers to a biological substrate outside of a volume or surface occupied by a therapeutic target. Such therapeutic targets include, but are not limited to, a tumor, a volume of infected tissue, a volume of degenerated tissue, a volume of inflamed tissue, a blood clot, or a region of plaque.

The term “pharmaceutical combination” as used herein, means a product that results from the mixing or combining of more than one active ingredient and includes both fixed and non-fixed combinations of the active ingredients. The term “fixed combination” means that the active ingredients, e.g. metal nanoparticles described herein and a co-agent, are both administered to a patient simultaneously in the form of a single entity or dosage. The term “non-fixed combination” means that the active ingredients, e.g. metal nanoparticles described herein and a co-agent, are administered to a patient as separate entities either simultaneously, concurrently or sequentially with no specific intervening time limits, wherein such administration provides effective levels of the two agents in the body of the patient. The latter also applies to cocktail therapy, e.g. the administration of three or more active ingredients.

A “subject,” as referred to herein, can be any vertebrate, though preferably a mammal (e.g., a mouse, rat, cat, guinea pig, hamster, rabbit, zebrafish, dog, non-human primate, or human) unless specified otherwise.

The term “therapeutic target” refers to a biological substrate (e.g., a tumor, a region of infected tissue, or a region of atheromatous plaque) that is to be acted upon by metal nanoparticles as described herein.

The terms “treat,” “treating” or “treatment,” as used herein, include alleviating, abating or ameliorating symptoms or pathological indicia of a therapeutic target-associated disease or condition, (e.g., breast tumor-?breast cancer), preventing additional symptoms, ameliorating or preventing the underlying metabolic causes of symptoms, inhibiting the disease or condition, (e.g., arresting the development of the disease or condition), relieving the disease or condition, causing regression of the disease or condition, relieving a condition caused by the disease or condition, or stopping the symptoms of the disease or condition either prophylactically and/or therapeutically.

Throughout the specification, groups and substituents thereof can be chosen by one skilled in the field to provide stable moieties and compositions.

Other features, objects, and advantages will be apparent from the description and from the claims.

### Ni-NTA-Gold Clusters

In some embodiments a Ni-NTA-gold cluster exhibits the following advantages: 1) quantitative binding and forming stable (Ni-NTA-gold)-(His protein) complexes due to high affinity (His-protein is defined as a protein containing multiple histidine residues, e.g., 6 adjacent histidines); 2) site specific to engineered His-tag locations; 3) linkage to gold are short for higher resolution; 4) Binding can be reversed under mild conditions; e.g. reducing pH to 4.5 to protonate the histidines and disrupt their interactions with Ni-NTA-gold nanoparticles; using imidazole or chelating agents like EDTA to occupy the binding sites of NTAs; 5) binding under mild conditions, but also in high salt and in chaotropic agents. In further embodiments, these conditions are also used to eliminate unwanted interactions; 6) stability over wide pH range, high salt concentrations and various operations e.g. centrifugation, heating, concentration under reduced pressure and lyophilization; 7) gold detection tags allow for light and electron microscope, blots, lateral flow, ELISA detection and direct eye visualization; 8) high sensitivity when silver or gold metallographic enhancement is employed with gold nanoparticles.

Compared to antibody-gold labeling of a target protein, there are further advantages: 1) Higher labeling due to the much tighter binding; 2) The label is much smaller, since there is no antibody. IgG is 150 kD and ˜12 nm in size, plus the size of the gold. A small Ni-NTA-gold cluster (e.g., 2-3 times smaller) should lead to better penetration and labeling. 3) Higher resolution since no primary antibody and secondary antibody-gold need to be used. Typically an unlabeled primary antibody is used, followed by a gold-labeled secondary antibody. With the Ni-NTA-gold, this will bind in one step to the target antigen. 4) There is no IgG to denature. The Ni-NTA-gold cluster is stable to >80° C., whereas antibodies denature at 55° C., and are subject to proteolytic digestion and bacterial degradation. The Ni-gold preparation should be more active with a better shelf life. 5) No antibody needs to be produced or purchased. With modern molecular techniques, His-tag formation is routine; production of a new antibody, if it does not already exist, is more costly and many existing primary antibodies are expensive. 6) In a further embodiment, one universal label is used to detect many targets or proteins labeled with 6x-His, 5x-His or other multi sequential histidine residues, as opposed to antibody labeling where a different antibody is needed for each target protein. Unlike detection by anti-6xHis antibody, the 5 nm-Ni-NTA-gold detection does not require a specific location of the polyhistidine tag e.g. N- or C-terminus, and the presence of specific adjacent aminoacid sequences.

A 1.8 nm Ni-NTA-gold nanoparticle has previously been described (e.g. See Hainfeld et al., J Struct Biol. 1999 September;127(2):185-98). This material has become popular after several years of use. However, in accordance with the practice of the present disclosure, this particle has a number of shortcomings, including: a) it is very small and difficult to detect even by standard electron microscopy, b) its extinction coefficient is low, making it difficult to directly detect in assays by eye or a reader, c) it delivers a low amount of gold per labeled molecule, d) there is background or non-specific binding to some other non-His-tagged proteins or materials, and e) its x-ray absorption is low.

No one has tried to prepare a larger size of Ni-NTA-gold nanoparticle as (1) the 1.4 to 1.8 nm Ni-NTA-gold nanoparticle is continuously being used and (2) the preparation of such larger size nanoparticle often fails. Synthesis of larger 2-50 nm Ni-NTA-gold nanoparticles to that used in the preparation of the 1.8 nm Ni-NTA-gold nanoparticle has been met with a number of difficulties including aggregation, low activity, poor solubility, and high non-specific background.

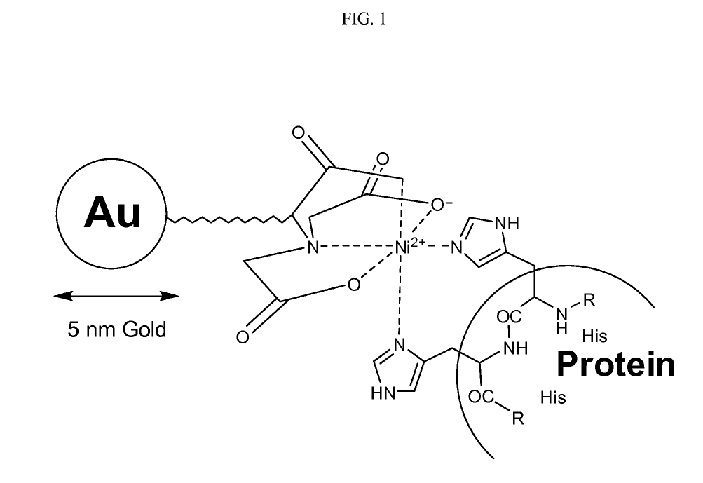

After many attempts and trial of various synthetic strategies, a 5 nm Ni-NTA-gold nanoparticle with the desired properties was produced. This nanoparticle showed no aggregation, excellent activity in binding His-tagged proteins, good solubility in aqueous buffers, low non-specific background and high stability over salt, heat and various operations, e.g. centrifugation, concentration under reduced pressure and lyophilization. The final formulation resulting in the combination of all these desirable properties was a surprising result when obtained. Its binding to a His-tagged protein is illustrated in FIG. 1.

In accordance with the present disclosure, the 5 nm Ni-NTA-gold nanoparticle overcomes the shortcomings of the small 1.8 nm Ni-NTA-gold nanoparticle: a) in one embodiment, it is larger and is easily detected by standard electron microscopy, even in negative stains, b) in another embodiment, it is large enough to be directly detected in assays by eye or a reader, c) in yet another embodiment, it delivers a high amount of gold per labeled molecule, d) in a further embodiment, there is low background or non-specific binding to non-His-tagged proteins or materials, and e) in yet a further embodiment, its x-ray absorption is high. Because the number of gold atoms goes up as the radius cubed, the number of gold atoms is increased significantly (20 times) since there are 200 gold atoms in a 1.8 nm cluster and 4,000 in a 5 nm cluster. In addition to these intrinsic properties, it was also surprisingly found that this 5 nm gold nanoparticle could serve as a nucleation site for deposition of additional metal, such as gold or silver. This additional deposition then increases the size further, thus making the particle even more detectable and useful.

Disclosed herein are compositions comprising a plurality of gold nanoparticles bound to at least one multidentate ligand such as a tetradentate ligand chelated to Nickel (II), wherein at least about 40% of the plurality of gold nanoparticles have an effective diameter about 5 nm±5% and methods of preparing same. In some embodiments, the compositions further comprise His-tagged proteins bound to the Nickel (II). In certain embodiments, the tetradentate ligand is selected from the group consisting of nitrilotriacetic acid (NTA), iminodiacetic acid, tris(carboxymethyl) ethylenediamine, ethylenediaminetetraacetic acid, diethylene triamine pentaacetic acid, DOTA (1,4,7,10-tetraazacyclododecane-1,4,7,10-tetraacetic acid), and the like. In certain embodiments, the tetradentate chelating group is nitrilotriacetic acid (NTA).

In another aspect, the present disclosure also provides compositions comprising a plurality of gold nanoparticle conjugates, comprising a plurality of gold nanoparticles bound to at least one multidentate ligand such as a tetradentate ligand chelated to Nickel (II), wherein at least about 90% of the plurality of gold nanoparticles have an effective diameter about 5 nm±25% and methods of preparing same.

In another aspect, compositions are in a dry powder form. In some embodiments, the dry powder form is free of salts, additives or stabilizers.

In another aspect, compositions are in a solution form. In certain embodiments, the compositions are in a form of a 0.5 μM concentration in 50 mM MOPS buffer solution.

In some embodiments, compositions are applicable to other shapes of metal nanoparticles including nanorods, nanoshells (metal with non-metal core), metal nanoparticle with hollow core, cubic, triangular, tetrahedral, and other shaped nanoparticles. In other embodiments, the methods are applicable to nanoparticles of various sizes, about 2 to about 1,000 nm, and even macro particles about 1 to about 50 μm.

In some embodiments, other metals besides Nickel (II) are used in the compositions including scandium, titanium, vanadium, chromium, manganese, iron, cobalt, copper, zinc, yttrium, zirconium, niobium, molybdenum, ruthenium, rhodium, palladium, silver, cadmium, mercury, hafnium, tantalum, tungsten, rhenium, osmium, iridium, platinum, gold, gadolinium, aluminum, gallium, indium, tin, thallium, lead, bismuth, magnesium, calcium, strontium, barium, lithium, sodium, potassium, boron, silicon, phosphorus, germanium, arsenic, antimony, and combinations thereof. In certain embodiments, the uses are optimized by optimal combinations of the nanoparticle components. For example, in some embodiment for MRI imaging, gadolinium is used.

In some embodiments, compositions are used, for example without limitation, for high resolution molecular labeling for electron and light microscopy, as a signal for detection, for specific binding to a target molecule, or for constructing nanoparticle conjugates with macromolecules and other materials. In some embodiments, compositions are used in diagnostic tests including, for example without limitation, lateral flow, ELISA, dot blots, chips, and other formats. In some embodiments, compositions are also used to provide imaging of various targets in vivo by various technologies, for example without limitation, x-ray CT or planar x-rays, MRI, PET, SPECT, and the like. In some embodiments, compositions are also used for therapies including, for example without limitation, enhancement of radiotherapy and drug delivery.

In some embodiments, in each nanoparticle at least about 50% (including about 50%, about 60%, about 70%, about 80%, about 90% and about 100%) of the core mass is made of one or more metals. In other embodiments, each of the nanoparticles consists essentially of one or more metals. In some embodiments, at least about 10% of the nanoparticles have a range of effective diameters from about 2 to about 1000 nm, (e.g., from about 3 to about 500 nm, from about 5 to about 200 nm or from about 5 to about 100 nm).

In some embodiments, compositions are used in rapid diagnostic kits. For example, one format of these kits is lateral flow, or “dipstick” tests similar to the home pregnancy tests. A drop of urine, blood, saliva, potentially contaminated water, or other analyte is placed on a strip that contains a primary antibody coupled to a detectable material. In some instances, the detectable material is, for example, a gold nanoparticle or a colored polymer such as latex. The antibody binds to the material to be tested for, (e.g., a protein, hormone, peptide, bacterium, virus, contaminant, pesticide, or other material). The antibody binds to the analyte (if it is present) and flows laterally down the strip. The analyte (if present) is captured by a test line of deposited primary antibody. The test is then read by eye or a reader. If the analyte was present, the gold nanoparticle or colored latex bead, for example, can be seen. Compositions described herein (e.g. 5 nm Ni-NTA-gold nanoparticles or conjugates) enable this test to be successful by providing a very detectable signal. It is superior to and different from the standard method since the antibody does not have to be chemically cross-linked to a gold nanoparticle, latex bead, or other substance. In some instances, if the antibody contains a His-tag it can be just mixed with the 5 nm Ni-NTA-gold nanoparticle to form a stable conjugate. In some embodiments, any protein or material that binds to the analyte desired to detect is used. For some instances, a His-tag is programmed in to a protein which then is manufactured by recombinant technology. In other instances, a His-tag is added to another molecule by cross-linking it with a multiple histidine containing peptide. In other instances a peptide that binds the analyte contains histidine residues that then bind the Ni-NTA-5 nm gold nanoparticles.

There are many detection modalities for compositions (e.g. 5 nm Ni-NTA-gold nanoparticles or conjugates). In some instances, the signal is read by eye, or by a reader. In some instances, enhancement is used with catalytic deposition of additional metals such as gold, silver, and copper. In some embodiments, compositions are also detected by establishing electrical conduction between electrodes or change of electrical properties (e.g., impedance, capacitance, frequency response). In some instances, various light methods such as reflection, dark field, scattering, and absorbance are used. In some instances, detection is by x-rays, interaction with particle beams, electromagnetic waves, alteration of sonication and magnetic resonance imaging patterns or properties. Formats of detection are varied and, in some embodiments, include lateral flow devices, dot blots, ELISA assays, cell binding assays, light, electron, atomic force, scanning tunneling microscopies, near field optics, lasers, and the like.

The compositions described herein (e.g. 5 nm Ni-NTA-gold nanoparticles or conjugates) basically enable rapid, stable, and quantitative coupling to histidine tagged or containing molecules. In some instances, this binding is made even more active and stable by designing in multiple NTA groups into the nanoparticle. In an antibody assay, for example, there are typically two steps: the application of the primary antibody, which detects the target, followed by a secondary antibody that is coupled to a detection moiety. Use of compositions such as a 5 nm Ni-NTA-gold nanoparticle considerably improves this procedure. Both primary and secondary antibody coupled to a detection moiety are eliminated and replaced by the binding and detection of 5 nm Ni-NTA-gold nanoparticle. This not only eliminates preparation of the secondary antibody-detection moiety conjugate and the use of expensive primary antibody, but enhances the binding to the targets, since the binding constant is higher than a secondary antibody. Furthermore, compositions such as a 5 nm Ni-NTA-gold nanoparticle reagent are more stable than an antibody; it does not require refrigeration and is impervious to enzymatic breakdown and less susceptible to bacterial degradation. It can also be dried for storage. Compositions (e.g. a 5 nm Ni-NTA-gold nanoparticle or conjugates), in some embodiments, therefore are used in many diagnostic tests, or anywhere that the binding protein, peptide, nucleic acid, lipid, drug, organic compound, synthetic analogs, or substance contains a multiple histidine component.

Other applications include use of delivering targeting agents in vivo. For example, an antibody, protein, peptide, nucleic acid, lipid, drug that that binds to a target tissue to be detected can be labeled with compositions (e.g. 5 nm Ni-NTA-gold nanoparticles or conjugates) to provide a signal. The signal is detected, in some instances, by x-ray absorption, visible light, thermal heating and detection by infrared application, change in electrical tissue properties, or other means. Examples include detection of tumors by antibodies or peptides directed to them labeled with compositions (e.g., 5 nm Ni-NTA-gold nanoparticles or conjugates), detection of atherosclerotic plaque, deep vein thrombosis, damaged heart tissue, blood clots, tissue morphology, and the like. X-ray absorption of gold is excellent and provides high resolution imaging therapy.

### Diagnosis and Therapy Applications

In some embodiments, compositions (e.g., Ni-NTA-5 nm gold nanoparticles or conjugates) are used in diagnostic applications including, but not limited to, lateral flow and microscopic examinations. The exemplary Ni-NTA-5 nm gold nanoparticles bind and detect polyhistidine-tagged materials including antibody, peptides, proteins, or other molecules which target or bind to tumor antigens, viral antigens, receptors, proteins, lipids, carbohydrates, nucleic acids, pesticides, and other chemicals and materials. The gold nanoparticle is detectable by various means, including light scattering, silver, gold and other metal enhancement (catalytic deposition), light, electron, and scanning probe microscopies, direct visualization, colorimetric absorption, modification of electrical or fluorescent properties, and x-ray fluorescence. Tests and test kits can be useful in the diagnosis of cancer, viral infection and other diseases and conditions. Furthermore, gold nanoparticles absorb x-rays well, and the exemplary Ni-NTA-5 nm gold nanoparticles, in some embodiments, are used as imaging contrast agents.

In some embodiments, compositions (e.g., Ni-NTA-5 nm gold nanoparticles or conjugates) are also used in therapeutic applications. Besides what has been disclosed herein, one skilled in the art will readily perceive many other such applications. In some instances, compositions are used during radiotherapy of cancer to absorb beam energy and deposit it in the tumor region, thus increasing the local dose. For example, a His-tagged antibody that targets the tumor or tumor vasculature is labeled with Ni-NTA-5 nm gold nanoparticles or conjugates. After localization to the tumor, therapeutic x-rays are applied. Various other sources may be employed, including, but not limited to: electrons, protons, ion beams such as beams of carbon ions, and neutrons.

In some embodiments, compositions (e.g., 5 nm gold nanoparticles or conjugates) absorb radiation in the ultraviolet, visible, and near infrared regions. In certain embodiments, infrared absorption is greatly enhanced by aggregating or placing in close proximity compositions such as Ni-NTA-5 nm gold nanoparticles. This can be accomplished by loading multiple particles onto closely spaced His tags, or allowing the particles to aggregate in the endosome and lysosome. Upon irradiation with an infrared source, the particles heat up and can send nearby cells into apoptosis or necrosis. In certain embodiments, tumors, and the like are treated this way.

### Novel Nanomaterials

In some embodiments, new constructs are formed with novel properties by combining compositions (e.g., Ni-NTA-5 nm gold nanoparticles or conjugates) with other materials. In certain embodiments, compositions (e.g., Ni-NTA-5 nm gold nanoparticles or conjugates) are added to proteins expressed with the His tag (multiple histidines). For example, a 6x-His-tag can be expressed on the capsomeres of a virus, and then labeled with Ni-NTA-5 nm gold nanoparticles. In some instances, this forms an icosohedron with gold nanoparticles arranged in a definite pattern on the virus surface.

By placing the gold nanoparticles close together, as is done in these examples, the absorption spectrum red shifts and the constructs become more absorbent in the near infrared region, making the constructs useful for heating by an infrared source.

Surfaces can also be labeled with proteins or peptides containing the His tag. In some embodiments, these are then linked to form novel patterns by incubating with composition such as Ni-NTA-5 nm gold nanoparticles or conjugates. In some instances, these are useful in biosensors and batteries.

In some embodiments, compositions provided herein (e.g., Ni-NTA-5 nm gold nanoparticles or conjugates) have bound to them functional groups that upon irradiation of the nanoparticle are released and/or activated. Functional groups that can be released include, but are not limited to, alkylating agents, antibiotics, cytokines, anti-cancer agents, thermosensitive liposomes, bioactive peptides, drugs, anti-fungal agents, radioactive elements, enzymes, nucleic acids, hormones, and imaging agents.

Functional groups that can be activated by irradiation include, but are not limited to, compounds that generate free radicals, compounds that ionize, prodrugs that are converted into active drugs, proenzymes that are converted into enzymes, and unreactive compounds that are converted into active ones. The released or activated materials can be used for therapy, or in industrial or other processes, for example to initiate polymerization and control chemical reactions.

Certain activated compounds can be used to degrade the nanoparticles and thereby enhance their clearance. For example, small gold nanoparticles are broken down by cyanides and small molecule thiols, and these could be created from other compounds (such as unreactive disulfides) by irradiation. Since release and/or activation is controlled by the irradiation (e.g., UV irradiation), the irradiation can be metered or applied at different times to achieve a time release from a reservoir of the nanoparticles (e.g., over a period of minutes to months). Two or more reactants can be carried on the shell of one nanoparticle in close proximity where a reaction between the reactants only occurred upon energy absorption and emission by the nanoparticle. Irradiation sources to stimulate these processes include, but are not limited to: IR, ionizing radiation, visible light, microwaves, radio frequency, and ultrasound.

In the case of IR irradiation, metal nanoparticles can be heated by IR illumination either continuously or pulsed to greater than body temperatures and also to very high temperatures (100° C.-1000° C.). The high heat achievable at the nanoparticle surface can be used to perform chemical reactions that would not otherwise occur. By appropriate choice of the linkers and reactive groups, bond scission, bond formation, and creation of free radicals and ions can be achieved. These chemical reactions can be used to either activate functional groups or release them from the nanoparticle.

Where the ionizing radiation is X-ray radiation, metal nanoparticles, in some embodiments, produce fluorescent photons, secondary electrons, electron-positron pairs, and Auger electrons. Auger electrons are very appropriate for the use disclosed here because they are short range, typically traveling only 1-20 nm in tissue-like material. The effect of these electrons, however, is great in that range, and they can cause ionizations, bond breakage, and free radical formation. Higher atomic number containing nanoparticles (with atomic number>25) have useful yields of Auger electrons upon x-ray irradiation. Auger electrons are useful for activating or releasing therapeutic or other compounds.

For example, in some embodiments, nanoparticle-bound porphyrins are activated by Auger electrons, and nanoparticle-bound anti-cancer agents are released by this mechanism. In some embodiments, hydroxyl radicals from hydroxyl groups are produced and liberated from a nanoparticle-bound therapeutic agent. These and other free radicals, in other embodiments, are then diffused from the nanoparticle and travel up to several microns, thus extending the effective therapeutic range of the nanoparticle. For example, a nanoparticle at a cell surface can be a source of free radicals that diffuse into the nucleus of a cell, altering its DNA, thus killing it.

Overall, this approach also has the advantage that the activation and/or release only occur where the irradiation is directed. This is in contrast to drugs that are typically administered systemically which lead to toxicity in other tissues and organs.

In some embodiments, the metal nanoparticles or conjugates are functionalized with a therapeutic agent (e.g., an anticancer agent or a thrombolytic agent) bound to the nanoparticles or conjugates through a photocleavable linker. Photocleavable linkers are linkers that are cleaved upon exposure to light (see, e.g., Goldmacher et al. (1992) Bioconj. Chem. 3:104-107) thereby releasing the targeted agent (e.g., a linked anti-cancer agent) upon exposure to light. Photocleavable linkers that are cleaved upon exposure to light are known. See, e.g., Ottl et al. (1998), Bioconjug. Chem., 2:143-151; Ottl (1998), Methods Enzymol 291:155-175; Yan et al (2004), Bioconjug. Chem., 15(5):1030-1036; and Kim et al. (2006), Bioorg Med Chem Lett., 16(15):4007-4010.

In some embodiments, the metal nanoparticles or conjugates are functionalized so as to associate with their surface an antibody, a stealth group, a thermosensitive liposome, a peptide, a polypeptide (e.g., a thermophilic enzyme), a nucleic acid, a drug, an organic moiety, a fluorophore, a carbohydrate, a lipid, or any combination thereof. Each of these can be associated either directly with the surface of the metal nanoparticle (e.g., through a sulfhydryl moiety) or indirectly through a bifunctional crosslinker or organic shell coating the surface of the metal nanoparticle. Methods for derivatizing metal nanoparticles are known in the art. See, e.g., Daniel et al. (2004), Chem. Rev., 104:293-346. See also U.S. patent application Ser. Nos. 11/271,392 and 11/549,071.

In some embodiments, the metal nanoparticles or conjugates are functionalized with an antibody that binds, e.g., a tumor or tumor-associated antigen, including cancer-germ cell (CG) antigens (MAGE, NY-ESO-1), mutational antigens (MUM-1, p53, CDK-4), over-expressed self-antigens (p53, HER2/NEU), viral antigens (from Papilloma Virus, Epstein-Barr Virus), tumor proteins derived from non-primary open reading frame mRNA sequences (Y-ESO1, LAGE1), Melan A, MART-1, MAGE-1, MAGE-3, BAGE, GAGE-1, GAGE-2, tyrosinase, gp100, gp75, HER-2/neu, c-erb-B2, CEA, PSA, MUC-1, CA-125, Stn, TAG-72, KSA (17-1A), PSMA, p53 (point mutated and/or over expressed), RAS (point mutated), HER-1/EGFR, calcitonin, cancer antigen 19-9, cancer antigen 125, alpha-fetoprotein, S-100 antigen, TA-90, antigen, VEGF, GD2, GM2, GD3, Anti-Id, CD20, CD19, CD22, CD36, Aberrant class II, B1, CD25 (IL-2R) (anti-TAC), or HPV. Metal nanoparticle-associated antibodies are useful, e.g., to direct and localize nanoparticles to a therapeutic target (e.g., a tumor) or an analyte ex vivo. In one embodiment, the antibody is a humanized antibody.

In other embodiments, the metal nanoparticles or conjugates are combined with a stealth group, e.g., polyethylene glycol (PEG), a PEG derivative, a poly(amino)acid, e.g., poly(hydroxy-L-asparagine (Romberg et al. (2006), Biochim Biophys Acta, (2007 March;1768(3):737-43), a biodegradable, biocompatible polymer, e.g., poly(lactic-co-glycolic acid)(PLGA), a carbohydrate, or a polypeptide.

In other embodiments, the metal nanoparticles or conjugates are functionalized with a thermophilic enzyme.

In one embodiment, the therapeutic target is provided metal nanoparticles or conjugates that are functionalized with a thermophilic enzyme that has significant activity only at supraphysiological temperatures (e.g., 60-85° C.). The therapeutic target is subsequently exposed to a dose of infrared radiation to increase the temperature of the therapeutic target in the presence of a substrate for the metal nanoparticle-bound thermophilic enzyme. For example, the thermophilic enzyme (e.g., β-galactosidase from Thermotoga maritima can be used in conjunction with, e.g., an anti-cancer pro-drug such as galactose-geldaymycin. See Cheng et al. (2005), J. Med. Chem., 48(2):645-652. Thus, conversion of the cancer pro-drug by the thermophilic enzyme will be localized to regions of elevated temperature within the therapeutic target. Accordingly, cells in a therapeutic target tissue can be killed either directly by heat ablation or indirectly by heat-driven enzymatic conversion of a pro-drug into an active cytotoxic agent. In some embodiments, the activated agent or enzyme can be used to locally produce or modulate other biological or chemical effects. For example, thermophilic, fibrinolytic enzymes, e.g., subtilisin, can dissolve blood clots, or other enzymes can be activated to break down inflammatory tissue, atherosclerotic plaque, neurofibrillary tangles, plaque associated with Alzheimer's and neurodegenerative or other diseases, enzymatic fat catabolism to reduce obesity and atheromas, metalloproteinases to break down cell barriers, or enzymes to accelerate chemical processes.

It is also possible to make use of a metal nanoparticle-linked thermophilic enzyme in combination with a marker substrate to transiently activate the substrate during infrared heating of nanoparticle aggregates and thereby “mark” cells in a therapeutic target (e.g., a tumor). This is useful, e.g., to track cells that “escape” from a therapeutic target (i.e., even after treatment) as can occur in, e.g., metastasis of a tumor. For example, the β-galactosidase substrate 2-Fluoro-4-nitrophenol-beta-D-galactopyranoside has been used to track cells expressing β-galactosidase in vivo by magnetic resonance imaging. See Kodibagkar et al. (2006), Mag. Res. Im., 24(7):959-962. The in vivo β-galactosidase substrate, DDAOG, a conjugate of beta-galactoside and 7-hydroxy-9H-(1,3-dichloro-9,9-dimethylacridin-2-one) (DDAO), has been used to image β-galactosidase-expressing glioma cells in vivo by far red fluorescence imaging. See Tung et al. (2004), Cancer Res., 64(5): 1579-1583.

A wide variety of thermophilic enzymes are known in the art. See, e.g., Vielle et al. (2001), Microb. And Mol. Suitable thermophilic enzymes include, but are not limited to, thermophilic alkaline phosphatases (e.g., from T. neapolitana), β-galactosidases (e.g., from T. maritima), proteases (e.g., WF146 protease), endoglucanases (e.g., from T. maritima), or DNA polymerases (e.g., Taq polymerase). Metal nanoparticle-associated thermophilic enzymes are useful, e.g., for heat-dependent enzymatic conversion of a pro-drug (e.g., galactose-geldanamycin conjugates) within or in close proximity to a therapeutic target.

In further embodiments, the metal nanoparticles or conjugates are functionalized with a thermosensitive liposome. Thermosensitive liposomes as referred to herein undergo a gel-to-liquid crystalline phase transition at temperatures higher than normal human physiological temperatures, e.g., temperatures from about 38° C. to about 45° C.), and thereby release any solutes (e.g., an anti-cancer agent) entrapped within the liposome into the surrounding solution. Thus, infrared heating of aggregates of metal nanoparticles having bound thermosensitive liposomes can be used to locally release therapeutic agents contained in the thermosensitive liposomes. Examples of thermosensitive liposomes, their synthesis, and their use are described in, e.g., U.S. Pat. Nos. 6,200,598, 6,623,430, and 6,690,976.

In some embodiments, the thermosensitive liposomes contain an anti-cancer agent, (e.g., a radiosensitizer agent such as, 5-Iododeoxyuridine, cisplatin, or Efaproxiral).

In other embodiments, the thermosensitive liposomes contain a nucleic acid, (e.g., a single or double stranded oligonucleotide). For example, the oligonucleotides can be antisense or RNAi molecules. In one embodiment, the thermosensitive liposome contains anti-angiogenic RNAi molecules. For example, the anti-angiogenic RNAi can be an anti-VEGF RNAi as described in, e.g., U.S. Pat. No. 7,148,342 or in U.S. patent application Ser. No. 11/340,080. In further embodiments, the thermosensitive liposomes contain a polypeptide. For example, the polypeptide can be a protein having thrombolytic activity such as, reteplase (r-PA or Retavase), alteplase (t-PA or Activase), urokinase (Abbokinase), prourokinase, anisoylated purified streptokinase activator complex (APSAC), and streptokinase.

In yet other embodiments, the thermosensitive liposomes contain, a fluorophore, preferably an infrared fluorophore. Localized release of the fluorophore from thermosensitive liposomes in vivo (e.g., within a tumor) can be used to label cells at a particular site and point in time to subsequently track their location in vivo. For example, tumor cells or clusters of tumor cells that are fluorescently “tagged” at time of a treatment, but survive the treatment can be tracked should they metastasize to other regions. Many suitable fluorophores are known in the art. See, e.g., “The Handbook-A Guide to Fluorescent Probes and Labeling Technologies,” Molecular Probes, Inc., Eugene, Oreg., (2004). For example, polypeptides can be labeled with one or more of the following fluorophores: 7-amino-4-methylcoumarin-3-acetic acid (AMCA), Texas Red™ (Molecular Probes, Inc., Eugene, Oreg.), 5-(and-6)-carboxy-X-rhodamine, lissamine rhodamine B, 5-(and-6)-carboxyfluorescein, fluorescein-5-isothiocyanate (FITC), 7-diethylaminocoumarin-3 carboxylic acid, tetramethylrhodamine-5-(and-6)-isothiocyanate, 5-(and-6)-carboxytetramethylrhodamine, 7-hydroxycoumarin-3-carboxylic acid, 6-[fluorescein 5(and-6)-carboxamido]hexanoic acid, N-(4,4-difluoro-5,7-dimethyl-4-bora-3a,4a diaza-3-indacenepropionic acid, eosin-5-isothiocyanate, erythrosin-5-isothiocyanate, phycoerythrin (B-, R-, or cyanine-), allophycocyanin, Oregon Green™, Cascade™ blue acetylazide, Alexa Fluor Dyes™ (Molecular Probes, Inc., Eugene, Oreg.), cyanine dyes, e.g., Cy3™, Cy5™ and Cy7™ dyes (Amersham Biosciences, UK, LTD), and near infrared cyanine fluorochromes as described in Lin et al. (2002), Bioconjugate Chem., 13:605-610. In one embodiment, the fluorophore is IR-786. See Flaumenhaft et al. (2007), Circulation, 115(1):84-93. In another embodiment the fluorophore is IR-Dye78. See Zaheer et al. (2002), Mol. Imaging, 1(4):354-364. See also U.S. patent application Ser. No. 11/149,602.

### Pharmaceutical Formulations of Metal Nanoparticle Compositions

Pharmaceutical compositions that include the metal nanoparticles described herein may be formulated in a conventional manner using one or more physiologically acceptable carriers including excipients and auxiliaries which facilitate processing of the metal nanoparticles into preparations which can be used pharmaceutically. Proper formulation is dependent upon the route of administration chosen. Well-known techniques, carriers, and excipients may be used as suitable and as understood in the art. A summary of pharmaceutical compositions described herein may be found, for example, in Remington: The Science and Practice of Pharmacy, Nineteenth Ed (Easton, Pa.: Mack Publishing Company, 1995); Hoover, John E., Remington's Pharmaceutical Sciences, Mack Publishing Co., Easton, Pa. 1975; Liberman, H. A. and Lachman, L., Eds., Pharmaceutical Dosage Forms, Marcel Decker, New York, N.Y., 1980; and Pharmaceutical Dosage Forms and Drug Delivery Systems, Seventh Ed. (Lippincott Williams & Wilkins 1999), herein incorporated by reference in their entirety.

Provided herein are pharmaceutical compositions that include metal nanoparticles or conjugates described herein, and a pharmaceutically acceptable, isotonicity agent(s), diluent(s), excipient(s), or carrier(s). In addition, the metal nanoparticles described herein can be administered as pharmaceutical compositions in which the metal nanoparticles are mixed with other active ingredients, as in combination therapy. In some embodiments, the pharmaceutical compositions may include other medicinal or pharmaceutical agents, carriers, adjuvants, such as preserving, stabilizing, wetting or emulsifying agents, solution promoters, salts for regulating the osmotic pressure, and/or buffers. In addition, the pharmaceutical compositions can also contain other therapeutically or diagnostically valuable substances such as anti-cancer agents, anti-inflammatory agents, thrombolytic agents, prodrugs, or in vivo enzyme marker substrates (e.g., 2-Fluoro-4-nitrophenol-beta-D-galactopyranoside).

In certain embodiments, compositions may also include one or more pH adjusting agents or buffering agents, including acids such as acetic, boric, citric, lactic, phosphoric and hydrochloric acids; bases such as sodium hydroxide, sodium phosphate, sodium borate, sodium citrate, sodium acetate, sodium lactate and tris-hydroxymethylaminomethane; and buffers such as citrate/dextrose, sodium bicarbonate and ammonium chloride. Such acids, bases and buffers are included in an amount required to maintain pH of the composition in an acceptable range.

In other embodiments, compositions may also include one or more salts in an amount required to bring osmolality of the composition into an acceptable range. Such salts include those having sodium, potassium or ammonium cations and chloride, citrate, ascorbate, borate, phosphate, bicarbonate, sulfate, thiosulfate or bisulfite anions; suitable salts include sodium chloride, potassium chloride, sodium thiosulfate, sodium bisulfite and ammonium sulfate.

In other embodiments, compositions may also include one or more isotonicity agents, such as dextrose, mannitol, or lactose.

The term “pharmaceutical combination” as used herein, means a product that results from the mixing or combining of more than one active ingredient and includes both fixed and non-fixed combinations of the active ingredients. The term “fixed combination” means that the active ingredients, e.g. a metal nanoparticle described herein and a co-agent, are both administered to a patient simultaneously in the form of a single entity or dosage. The term “non-fixed combination” means that the active ingredients, e.g. a metal nanoparticle described herein and a co-agent, are administered to a patient as separate entities either simultaneously, concurrently or sequentially with no specific intervening time limits, wherein such administration provides effective levels of the two agents at a therapeutic target in the body of the patient. The latter also applies to cocktail therapy, e.g. the administration of three or more active ingredients.

A pharmaceutical composition, as used herein, refers to a mixture of metal nanoparticles described herein, with other chemical components, such as carriers, stabilizers, diluents, dispersing agents, suspending agents, thickening agents, and/or excipients. The pharmaceutical composition facilitates providing metal nanoparticles to a therapeutic target. In practicing the methods of treatment or use provided herein, therapeutically effective amounts of metal nanoparticles described herein are administered in a pharmaceutical composition to a subject having a disease, disorder, or condition to be treated. Preferably, the subject is a human. A therapeutically effective amount can vary widely depending on the severity of the disease, the age and relative health of the subject, the physical characteristics of the metal nanoparticles used, and other factors. The metal nanoparticles described herein can be used alone or in combination with one or more therapeutic agents as components of mixtures.

The pharmaceutical formulations described herein can be administered to a subject by multiple administration routes, including parenteral (e.g., intravenous, subcutaneous, intramuscular), topical, rectal, or transdermal administration routes. The pharmaceutical formulations described herein include, but are not limited to, aqueous liquid dispersions, self-emulsifying dispersions, solid solutions, liposomal dispersions, or solid dosage forms.

The pharmaceutical compositions will include at least one metal nanoparticle described herein, such as, for example, a gold nanoparticle functionalized with anti-tumor antigen antibody.

The term “acceptable” with respect to a formulation, composition or ingredient, as used herein, means having no persistent detrimental effect on the general health of the subject being treated.

As used herein, amelioration or palliation of the symptoms of a particular disease, disorder or condition by administration of a particular pharmaceutical composition refers to any lessening of severity, delay in onset, slowing of progression, or shortening of duration, whether permanent or temporary, lasting or transient that can be attributed to or associated with administration of the composition.

“Antifoaming agents” reduce foaming during processing which can result in coagulation of aqueous dispersions, bubbles in the finished film, or generally impair processing. Exemplary anti-foaming agents include silicon emulsions or sorbitan sesquoleate.

“Antioxidants” include, for example, butylated hydroxytoluene (BHT), sodium ascorbate, ascorbic acid, sodium metabisulfite and tocopherol. In certain embodiments, antioxidants enhance chemical stability where required.

In certain embodiments, compositions provided herein may also include one or more preservatives to inhibit microbial activity. Suitable preservatives include mercury-containing substances such as merfen and thiomersal; stabilized chlorine dioxide; and quaternary ammonium compounds such as benzalkonium chloride, cetyltrimethylammonium bromide and cetylpyridinium chloride.

“Bioavailability” refers to the percentage of the weight of metal nanoparticles disclosed herein that is delivered into a therapeutic target. The total exposure (AUC(0-∞)) of a drug when administered intravenously is usually defined as 100% bioavailable (F %).

“Carrier materials” include any commonly used excipients in pharmaceutics and should be selected on the basis of compatibility with metal nanoparticles described herein. Exemplary carrier materials include, e.g., binders, suspending agents, disintegration agents, filling agents, surfactants, solubilizers, stabilizers, lubricants, wetting agents, diluents, and the like. “Pharmaceutically compatible carrier materials” may include, but are not limited to, acacia, gelatin, colloidal silicon dioxide, calcium glycerophosphate, calcium lactate, maltodextrin, glycerine, magnesium silicate, polyvinylpyrrollidone (PVP), cholesterol, cholesterol esters, sodium caseinate, soy lecithin, taurocholic acid, phosphotidylcholine, sodium chloride, tricalcium phosphate, dipotassium phosphate, cellulose and cellulose conjugates, sugars sodium stearoyl lactylate, carrageenan, monoglyceride, diglyceride, pregelatinized starch, and the like. See, e.g., Remington: The Science and Practice of Pharmacy, Nineteenth Ed (Easton, Pa.: Mack Publishing Company, 1995); Hoover, John E., Remington's Pharmaceutical Sciences, Mack Publishing Co., Easton, Pa. 1975; Liberman, H. A. and Lachman, L., Eds., Pharmaceutical Dosage Forms, Marcel Decker, New York, N.Y., 1980; and Pharmaceutical Dosage Forms and Drug Delivery Systems, Seventh Ed. (Lippincott Williams & Wilkins 1999).

“Dispersing agents,” and/or “viscosity modulating agents” include materials that control the diffusion and homogeneity of metal nanoparticles through liquid media or a granulation method or blend method. In some embodiments, these agents also facilitate the effectiveness of a coating or eroding matrix. Exemplary diffusion facilitators/dispersing agents include, e.g., hydrophilic polymers, electrolytes, Tween® 60 or 80, PEG, polyvinylpyrrolidone (PVP; commercially known as Plasdone®), and the carbohydrate-based dispersing agents such as, for example, hydroxypropyl celluloses (e.g., HPC, HPC-SL, and HPC-L), hydroxypropyl methylcelluloses (e.g., HPMC K100, HPMC K4M, HPMC K15M, and HPMC K100M), carboxymethylcellulose sodium, methylcellulose, hydroxyethylcellulose, hydroxypropylcellulose, hydroxypropylmethylcellulose phthalate, hydroxypropylmethylcellulose acetate stearate (HPMCAS), noncrystalline cellulose, magnesium aluminum silicate, triethanolamine, polyvinyl alcohol (PVA), vinyl pyrrolidone/vinyl acetate copolymer (S630), 4-(1,1,3,3-tetramethylbutyl)-phenol polymer with ethylene oxide and formaldehyde (also known as tyloxapol), poloxamers (e.g., Pluronics F68®, F88®, and F108®, which are block copolymers of ethylene oxide and propylene oxide); and poloxamines (e.g., Tetronic 908®, also known as Poloxamine 908®, which is a tetrafunctional block copolymer derived from sequential addition of propylene oxide and ethylene oxide to ethylenediamine (BASF Corporation, Parsippany, N.J.)), polyvinylpyrrolidone K12, polyvinylpyrrolidone K17, polyvinylpyrrolidone K25, or polyvinylpyrrolidone K30, polyvinylpyrrolidone/vinyl acetate copolymer (S-630), polyethylene glycol, e.g., the polyethylene glycol can have a molecular weight of about 300 to about 6000, or about 3350 to about 4000, or (about 4000 to about 5400, sodium carboxymethylcellulose, methylcellulose, polysorbate-80, sodium alginate, gums, such as, e.g., gum tragacanth and gum acacia, guar gum, xanthans, including xanthan gum, sugars, cellulosics, such as, e.g., sodium carboxymethylcellulose, methylcellulose, sodium carboxymethylcellulose, polysorbate-80, sodium alginate, polyethoxylated sorbitan monolaurate, polyethoxylated sorbitan monolaurate, povidone, carbomers, polyvinyl alcohol (PVA), alginates, chitosans and combinations thereof. Plasticizers such as cellulose or triethyl cellulose can also be used as dispersing agents. Dispersing agents particularly useful in liposomal dispersions and self-emulsifying dispersions are dimyristoyl phosphatidyl choline, natural phosphatidyl choline from eggs, natural phosphatidyl glycerol from eggs, cholesterol and isopropyl myristate.

Combinations of one or more erosion facilitator with one or more diffusion facilitator can also be used in the present compositions.

The term “diluent” refers to chemical compounds that are used to dilute metal nanoparticle compositions prior to administration. Diluents can also be used to stabilize nanoparticle compositions. Salts dissolved in buffered solutions (which also can provide pH control or maintenance) are utilized as diluents in the art, including, but not limited to a phosphate buffered saline solution. In certain embodiments, diluents increase bulk of the composition to facilitate compression or create sufficient bulk for homogenous blend for capsule filling. Such compounds include e.g., lactose, starch, mannitol, sorbitol, dextrose, microcrystalline cellulose such as Avicel®; dibasic calcium phosphate, dicalcium phosphate dihydrate; tricalcium phosphate, calcium phosphate; anhydrous lactose, spray-dried lactose; pregelatinized starch, compressible sugar, such as Di-Pac® (Amstar); mannitol, hydroxypropylmethylcellulose, hydroxypropylmethylcellulose acetate stearate, sucrose-based diluents, confectioner's sugar; monobasic calcium sulfate monohydrate, calcium sulfate dihydrate; calcium lactate trihydrate, dextrates; hydrolyzed cereal solids, amylose; powdered cellulose, calcium carbonate; glycine, kaolin; mannitol, sodium chloride; inositol, bentonite, and the like.

“Absorption” typically refers to the process of movement of metal nanoparticles from a site of administration into a site of action in a therapeutic target, e.g., metal nanoparticles extravasating from the general circulation into the interstitial space of a tumor.

“Pharmacodynamics” refers to the factors which determine the therapeutic efficacy observed relative to the concentration of metal nanoparticles at a therapeutic target site of action.

“Pharmacokinetics” refers to the factors which determine the attainment and maintenance of the appropriate concentration of metal nanoparticles at a therapeutic target site of action.

“Solubilizers” include compounds such as triacetin, triethylcitrate, ethyl oleate, ethyl caprylate, sodium lauryl sulfate, sodium doccusate, vitamin E TPGS, dimethylacetamide, N-methylpyrrolidone, N-hydroxyethylpyrrolidone, polyvinylpyrrolidone, hydroxypropylmethyl cellulose, hydroxypropyl cyclodextrins, ethanol, n-butanol, isopropyl alcohol, cholesterol, bile salts, polyethylene glycol 200-600, glycofurol, transcutol, propylene glycol, and dimethyl isosorbide and the like.

“Stabilizers” include compounds such as any antioxidation agents, buffers, acids, preservatives and the like.

“Suspending agents” include compounds such as polyvinylpyrrolidone, e.g., polyvinylpyrrolidone K12, polyvinylpyrrolidone K17, polyvinylpyrrolidone K25, or polyvinylpyrrolidone K30, vinyl pyrrolidone/vinyl acetate copolymer (S630), polyethylene glycol, e.g., the polyethylene glycol can have a molecular weight of about 300 to about 6000, or about 3350 to about 4000, or (about 4000 to about 5400, sodium carboxymethylcellulose, methylcellulose, hydroxypropylmethylcellulose, hydroxymethylcellulose acetate stearate, polysorbate-80, hydroxyethylcellulose, sodium alginate, gums, such as, e.g., gum tragacanth and gum acacia, guar gum, xanthans, including xanthan gum, sugars, cellulosics, such as, e.g., sodium carboxymethylcellulose, methylcellulose, sodium carboxymethylcellulose, hydroxypropylmethylcellulose, hydroxyethylcellulose, polysorbate-80, sodium alginate, polyethoxylated sorbitan monolaurate, polyethoxylated sorbitan monolaurate, povidone and the like.

“Surfactants” include compounds such as sodium lauryl sulfate, sodium docusate, Tween 60 or 80, triacetin, vitamin E TPGS, sorbitan monooleate, polyoxyethylene sorbitan monooleate, polysorbates, polaxomers, bile salts, glyceryl monostearate, copolymers of ethylene oxide and propylene oxide, e.g., Pluronic® (BASF), and the like. Some other surfactants include polyoxyethylene fatty acid glycerides and vegetable oils, e.g., polyoxyethylene (60) hydrogenated castor oil; and polyoxyethylene alkylethers and alkylphenyl ethers, e.g., octoxynol 10, octoxynol 40. In some embodiments, surfactants may be included to enhance physical stability or for other purposes.

“Viscosity enhancing agents” include, e.g., methyl cellulose, xanthan gum, carboxymethyl cellulose, hydroxypropyl cellulose, hydroxypropylmethyl cellulose, hydroxypropylmethyl cellulose acetate stearate, hydroxypropylmethyl cellulose phthalate, carbomer, polyvinyl alcohol, alginates, acacia, chitosans and combinations thereof.

“Wetting agents” include compounds such as oleic acid, glyceryl monostearate, sorbitan monooleate, sorbitan monolaurate, triethanolamine oleate, polyoxyethylene sorbitan monooleate, polyoxyethylene sorbitan monolaurate, sodium docusate, sodium oleate, sodium lauryl sulfate, sodium doccusate, triacetin, Tween 80, vitamin E TPGS, ammonium salts and the like.

The compositions described herein can be formulated for administration to a subject via any conventional means including, but not limited to, oral, parenteral (e.g., intravenous, subcutaneous, or intramuscular), buccal, intranasal, rectal or transdermal administration routes. As used herein, the term “subject” is used to mean any vertebrate, preferably a mammal, including a human or non-human. The terms patient and subject may be used interchangeably.

Formulations that include metal nanoparticles, suitable for intramuscular, subcutaneous, or intravenous injection may include physiologically acceptable sterile aqueous or non-aqueous solutions, dispersions, suspensions or emulsions, and sterile powders for reconstitution into sterile injectable solutions or dispersions. Examples of suitable aqueous and non-aqueous carriers, diluents, solvents, or vehicles include water, ethanol, polyols (propyleneglycol, polyethylene-glycol, glycerol, cremophor and the like), suitable mixtures thereof, vegetable oils (such as olive oil) and injectable organic esters such as ethyl oleate. Proper fluidity can be maintained, for example, by the use of a coating such as lecithin, by the maintenance of the required particle size in the case of dispersions, and by the use of surfactants. Formulations suitable for subcutaneous injection may also contain additives such as preserving, wetting, emulsifying, and dispensing agents. Prevention of the growth of microorganisms can be ensured by various antibacterial and antifungal agents, such as parabens, chlorobutanol, phenol, sorbic acid, and the like. It may also be desirable to include isotonicity agents, such as sugars (e.g., dextrose), mannitol, sodium chloride, and the like.

For intravenous injections, metal nanoparticle compositions described herein may be formulated in aqueous solutions, preferably in physiologically compatible buffers such as Hank's solution, Ringer's solution, or physiological saline buffer along with an isotonicity agent, (e.g., dextrose, mannitol, or lactose).

For other parenteral injections, appropriate formulations may include aqueous or nonaqueous solutions, preferably with physiologically compatible buffers or excipients. Such excipients are generally known in the art.

Parenteral injections may involve bolus injection or continuous infusion. Formulations for injection may be presented in unit dosage form, (e.g., in ampoules or in multi-dose containers, with an added preservative). The pharmaceutical compositions described herein may be in a form suitable for parenteral injection as a sterile suspensions, solutions or emulsions in oily or aqueous vehicles, and may contain formulatory agents such as suspending, stabilizing and/or dispersing agents. Pharmaceutical formulations for parenteral administration include aqueous solutions of the metal nanoparticles in water-soluble form. Additionally, suspensions of the metal nanoparticles may be prepared as appropriate oily injection suspensions. Suitable lipophilic solvents or vehicles include fatty oils such as sesame oil, or synthetic fatty acid esters, such as ethyl oleate or triglycerides, or liposomes. Aqueous injection suspensions may contain substances which increase the viscosity of the suspension, such as sodium carboxymethyl cellulose, sorbitol, or dextran. Optionally, the suspension may also contain suitable stabilizers or agents which increase the solubility of the metal nanoparticles to allow for the preparation of highly concentrated solutions. Alternatively, the active ingredient may be in powder form for constitution with a suitable vehicle, e.g., sterile pyrogen-free water, before use.

In some embodiments, the metal nanoparticles or conjugates described herein are administered topically and in other embodiments, formulated into a variety of topically administrable pharmaceutical compositions, such as solutions, suspensions, lotions, gels, pastes, balms, creams or ointments. Such pharmaceutical compositions in further embodiments contain solubilizers, stabilizers, tonicity enhancing agents, buffers and preservatives.

The transdermal dosage forms described herein may incorporate certain pharmaceutically acceptable excipients which are conventional in the art. In one embodiment, the transdermal formulations described herein include at least three components: (1) metal nanoparticles or conjugates; (2) a penetration enhancer; and (3) an aqueous adjuvant. In addition, transdermal formulations can include additional components such as, but not limited to, gelling agents, creams and ointment bases, and the like. In some embodiments, the transdermal formulation(s) further include a woven or non-woven backing material to enhance absorption and prevent the removal of the transdermal formulation from the skin. In other embodiments, the transdermal formulations described herein maintain a saturated or supersaturated state to promote diffusion into the skin.

Formulations suitable for transdermal administration of metal nanoparticles or conjugates described herein in some embodiments employ transdermal delivery devices and transdermal delivery patches and in other embodiments are lipophilic emulsions or buffered, aqueous solutions, dissolved and/or dispersed in a polymer or an adhesive. Such patches in some embodiments are constructed for continuous, pulsatile, or on demand delivery of pharmaceutical agents. Still further, transdermal delivery of the metal nanoparticles or conjugates described herein in other embodiments are accomplished by means of iontophoretic patches and the like. Conversely, absorption enhancers can be used to increase absorption. An absorption enhancer or carrier in other embodiments includes absorbable pharmaceutically acceptable solvents to assist passage through the skin. For example, transdermal devices are in the form of a bandage comprising a backing member, a reservoir containing the metal nanoparticles optionally with carriers, optionally a rate controlling barrier to deliver the metal nanoparticles or conjugates to the skin of the host at a controlled and predetermined rate over a prolonged period of time, and means to secure the device to the skin.

Transdermal formulations of the metal nanoparticle compositions described herein in other embodiments are administered using a variety of devices which have been described in the art. For example, such devices include, but are not limited to, U.S. Pat. Nos. 3,598,122, 3,598,123, 3,710,795, 3,731,683, 3,742,951, 3,814,097, 3,921,636, 3,972,995, 3,993,072, 3,993,073, 3,996,934, 4,031,894, 4,060,084, 4,069,307, 4,077,407, 4,201,211, 4,230,105, 4,292,299, 4,292,303, 5,336,168, 5,665,378, 5,837,280, 5,869,090, 6,923,983, 6,929,801 and 6,946,144. These references are incorporated by reference to the extent they are relevant.

The metal nanoparticles or conjugates described herein, in other embodiments are formulated in rectal compositions such as enemas, rectal gels, rectal foams, rectal aerosols, suppositories, jelly suppositories, or retention enemas, containing conventional suppository bases such as cocoa butter or other glycerides, as well as synthetic polymers such as polyvinylpyrrolidone, PEG, and the like. In suppository forms of the compositions, a low-melting wax such as, but not limited to, a mixture of fatty acid glycerides, optionally in combination with cocoa butter is first melted.

In some embodiments, the solid dosage forms disclosed herein are in the form of a tablet, (including a suspension tablet, a fast-melt tablet, a bite-disintegration tablet, a rapid-disintegration tablet, an effervescent tablet, or a caplet), a pill, a powder (including a sterile packaged powder, a dispensable powder, or an effervescent powder) a capsule (including both soft or hard capsules, e.g., capsules made from animal-derived gelatin or plant-derived HPMC, or “sprinkle capsules”), solid dispersion, solid solution, bioerodible dosage form, controlled release formulations, pulsatile release dosage forms, multiparticulate dosage forms, pellets, granules, or an aerosol. In other embodiments, the pharmaceutical formulation is in the form of a powder. In still other embodiments, the pharmaceutical formulation is in the form of a tablet, including but not limited to, a fast-melt tablet.

The pharmaceutical solid dosage forms described herein, in some embodiments, include metal nanoparticles or conjugates disclosed herein, and one or more pharmaceutically acceptable additives such as a compatible carrier, binder, filling agent, suspending agent, flavoring agent, sweetening agent, disintegrating agent, dispersing agent, surfactant, lubricant, colorant, diluent, solubilizer, moistening agent, plasticizer, stabilizer, penetration enhancer, wetting agent, anti-foaming agent, antioxidant, preservative, or one or more combination thereof.

### Methods of Dosing and Treatment Regimens

The metal nanoparticle or conjugates compositions described herein in further embodiments, are used in the preparation of medicaments for increasing the infrared absorptivity of a therapeutic target, or for the treatment of diseases or conditions that would benefit, at least in part, from increased infrared absorptivity of the therapeutic target. In addition, a method for treating any of the diseases or conditions described herein in a subject in need of such treatment, involves administration of pharmaceutical compositions containing metal nanoparticles described herein in therapeutically effective amounts to a therapeutic target in said subject.

The compositions containing the metal nanoparticles or conjugates described herein can be administered for prophylactic and/or therapeutic treatments. In therapeutic applications, the compositions are administered to a patient already suffering from a disease or condition, in an amount sufficient to cure or at least partially arrest the symptoms of the disease or condition. Amounts effective for this use will depend on the severity and course of the disease or condition, therapeutic target characteristics such as shape, volume, tissue depth, infrared irradiation dosage, and other factors such as previous therapy, the patient's health status, weight, and response to compositions in combination with infrared irradiation, as well as the judgment of the treating physician. It is considered well within the skill of the art for one to determine therapeutically effective amounts of therapeutic agents by routine experimentation (including, but not limited to, a dose escalation clinical trial).

In prophylactic applications, compositions containing the metal nanoparticles or conjugates described herein are administered to a patient susceptible to or otherwise at risk of a particular disease, disorder or condition associated with a therapeutic target. Such an amount is defined to be a “prophylactically effective amount or dose.” In this use, the precise amounts also depend on the patient's state of health, weight, and the like. It is considered well within the skill of the art for one to determine such prophylactically effective amounts by routine experimentation (e.g., a dose escalation clinical trial). When used in a patient, effective amounts for this use will depend on the severity and course of the disease, disorder or condition, previous therapy, the patient's health status and response to the drugs, and the judgment of the treating physician.

In general, however, doses employed for adult human treatment will typically be in the range of about 1-about 5 g/kg per administration, in some embodiments, about 10-about 800 mg/kg per administration. The desired dose in other embodiments is conveniently presented in a single dose or as divided doses administered simultaneously (or over a short period of time) or at appropriate intervals, for example as two, three, four or more sub-doses per day. In embodiments in which the nanoparticles are functionalized with a drug to be released or activated, the amount of the nanoparticle composition administered can be substantially less than that required for ablative tissue heating. For example, the dose in other embodiments is about 0.001-about 5 mg/kg. In other embodiments, where a combined pharmacological and thermal ablative effect is desired, intermediate dose ranges are used.

The foregoing ranges are merely suggestive, as the number of variables in regard to an individual treatment regime is large, and considerable excursions from these recommended values are not uncommon. Such dosages may be altered depending on a number of variables, not limited to the absorptive properties of the metal nanoparticles used, the therapeutic target to be treated, the disease or condition to be treated, the mode of administration, the requirements of the individual subject, the severity of the disease or condition being treated, and the judgment of the practitioner.

Toxicity and therapeutic efficacy of such therapeutic regimens can be determined by standard pharmaceutical procedures in cell cultures or experimental animals, including, but not limited to, the determination of the LD50 (the dose lethal to 50% of the population) and the ED₅₀ (the dose therapeutically effective in 50% of the population). The dose ratio between the toxic and therapeutic effects is the therapeutic index and it can be expressed as the ratio between LD₅₀ and ED50. Metal nanoparticle compositions exhibiting high therapeutic indices are preferred. The data obtained from cell culture assays and animal studies can be used in formulating a range of dosage for use in human. The dosage of such compositions lies preferably within a range of circulating concentrations that include the ED50 with minimal toxicity. The dosage may vary within this range depending upon the dosage form employed and the route of administration utilized. Doses may also be administered fractionally, i.e., with a regimen schedule over a period of days or weeks.

The compositions and methods described herein in other embodiments is used in conjunction with other well known therapeutic reagents that are selected for their particular usefulness against the condition that is being treated. In general, the compositions described herein and, in embodiments where combinational therapy is employed, other agents do not have to be administered in the same pharmaceutical composition, and may, because of different physical and chemical characteristics, have to be administered by different routes. The determination of the mode of administration and the advisability of administration, where possible, in the same pharmaceutical composition, is well within the knowledge of the skilled clinician. The initial administration can be made according to established protocols known in the art, and then, based upon the observed effects, the dosage, modes of administration and times of administration can be modified by the skilled clinician.

In certain instances, it is appropriate to administer metal nanoparticles or conjugates described herein in combination with another therapeutic agent. By way of example only, the benefit experienced by a patient is increased by administering one of the metal nanoparticle or conjugate compositions described herein with another therapeutic agent (e.g., an anti-cancer agent). In any case, regardless of the disease, disorder or condition being treated, the overall benefit experienced by the patient may simply be additive of the two therapeutic agents or the patient may experience a synergistic benefit.

The particular choice of therapeutic agents used will depend upon the diagnosis of the attending physicians and their judgment of the condition of the patient and the appropriate treatment protocol. The therapeutic agents (e.g., a metal nanoparticle composition and an anti-cancer compound) may be administered concurrently (e.g., simultaneously, essentially simultaneously or within the same treatment protocol) or sequentially, depending upon the nature of the disease, disorder, or condition, the condition of the patient, and the actual choice of therapeutic agents used. The determination of the order of administration, and the number of repetitions of administration of each therapeutic agent during a treatment protocol, is well within the knowledge of the skilled physician after evaluation of the disease being treated and the condition of the patient.

In some embodiments, therapeutically-effective dosages vary when the therapeutic agents are used in treatment combinations. Methods for experimentally determining therapeutically-effective dosages of therapeutic agents for use in combination treatment regimens are described in the literature. For example, the use of metronomic dosing, i.e., providing more frequent, lower doses in order to minimize toxic side effects, has been described extensively in the literature. Combination treatment further includes periodic treatments that start and stop at various times to assist with the clinical management of the patient.

For combination therapies described herein, dosages of the co-administered compositions will of course vary depending on the type of co-agents employed, on the disease or condition being treated and so forth. In addition, when co-administered with one or more biologically active agents, the metal nanoparticles provided herein may be administered either simultaneously with the other biologically active agent(s), or sequentially. If administered sequentially, the attending physician will decide on the appropriate sequence of administering protein in combination with the biologically active agent(s).

In any case, the multiple therapeutic agents (one of which is a metal nanoparticle described herein) may be administered in any order or even simultaneously. If simultaneously, the multiple therapeutic agents may be provided in a single, unified form, or in multiple forms (by way of example only, either as a single pill or as two separate pills). One of the therapeutic agents may be given in multiple doses, or both may be given as multiple doses. If not simultaneous, the timing between the multiple doses may vary from more than zero weeks to less than four weeks. In addition, the combination methods, compositions and formulations are not to be limited to the use of only two agents. The use of multiple therapeutic combinations is also envisioned.

It is understood that the dosage regimen to treat, prevent, or ameliorate the condition(s) for which relief is sought, can be modified in accordance with a variety of factors. These factors include the disorder from which the subject suffers, as well as the age, weight, sex, diet, and medical condition of the subject. Thus, the dosage regimen actually employed can vary widely and therefore can deviate from the dosage regimens set forth herein.

The pharmaceutical agents which make up the combination therapy disclosed herein may be a combined dosage form or in separate dosage forms intended for substantially simultaneous administration. The pharmaceutical agents that make up the combination therapy may also be administered sequentially, with either therapeutic agent being administered by a regimen calling for two-step administration. The two-step administration regimen may call for sequential administration of the active agents or spaced-apart administration of the separate active agents. The time period between the multiple administration steps may range from, a few minutes to several hours, depending upon the properties of each pharmaceutical agent, such as potency, solubility, bioavailability, plasma half-life and kinetic profile of the pharmaceutical agent. Circadian variation of the target molecule concentration may also determine the optimal dose interval.

In addition, the metal nanoparticle compositions described herein also may be used in combination with procedures that may provide additional or synergistic benefit to the patient. By way of example only, patients are expected to find therapeutic and/or prophylactic benefit in the methods described herein, wherein pharmaceutical compositions of a metal nanoparticle disclosed herein and/or combinations with other therapeutics are combined with genetic testing to determine whether that individual is a carrier of a mutant gene that is known to be correlated with certain diseases or conditions.

The compositions described herein and combination therapies can be administered before, during or after the occurrence of a disease or condition, and the timing of administering the composition containing metal nanoparticles can vary. Thus, for example, the compositions can be used as a prophylactic in order to prevent the occurrence of a disease or condition associated with a therapeutic target. The compositions can be administered to a subject during or as soon as possible after the onset of symptoms or after diagnosis. For acute conditions, the administration of the compositions can be initiated within the first 48 hours of the onset of symptoms for acute conditions, preferably within the first 48 hours of the onset of the symptoms, more preferably within the first 6 hours of the onset of the symptoms, and most preferably within 3 hours of the onset of the symptoms. The initial administration can be via any route practical, such as, for example, an intravenous injection, a bolus injection, infusion over 5 minutes to about 5 hours, a pill, a capsule, transdermal patch, buccal delivery, and the like, or combination thereof. Metal nanoparticle compositions are preferably administered as soon as is practicable after the onset of a disease or condition is detected or suspected, and for a length of time necessary for the treatment of the disease, such as, for example, either once or multiple treatments over about 5 days to about 6 months. The length of treatment can vary for each subject, and the length can be determined using the known criteria. For example, compositions containing the metal nanoparticles can be administered in combination with infrared radiation, repeatedly for at least 2 weeks, 1 month to about 5 years, or about 1 month to about 3 years.

### EXAMPLES

The following specific examples are to be construed as merely illustrative, and not limitative of the remainder of the disclosure in any way whatsoever. Without further elaboration, it is believed that one skilled in the art can, based on the description herein, utilize the present invention to its fullest extent. All publications cited herein are hereby incorporated by reference in their entirety. Where reference is made to a URL or other such identifier or address, it is understood that such identifiers can change and particular information on the internet can come and go, but equivalent information can be found by searching the internet. Reference thereto evidences the availability and public dissemination of such information.

### Example 1

### Synthesis of Ni-NTA-5 nm Gold Nanoparticles.

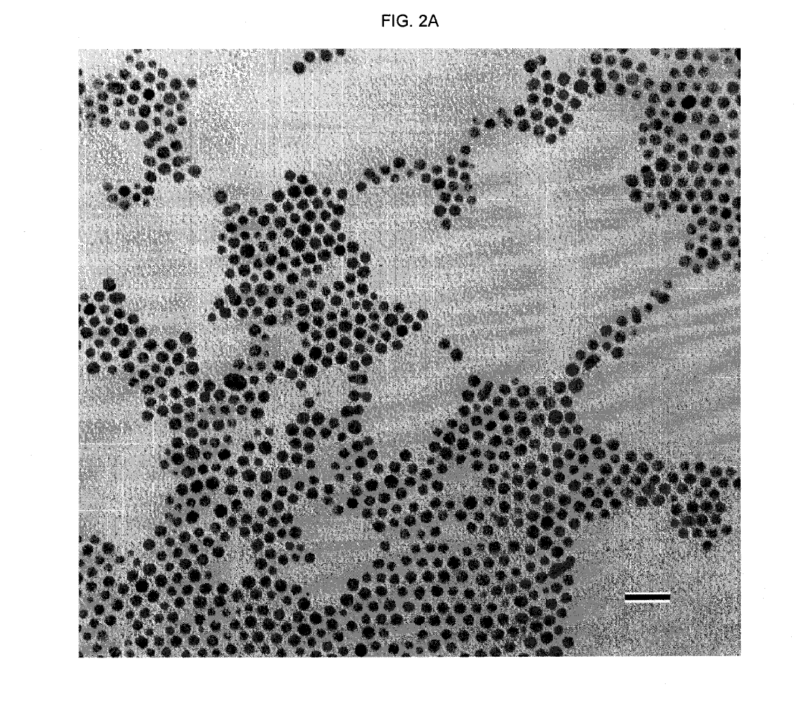

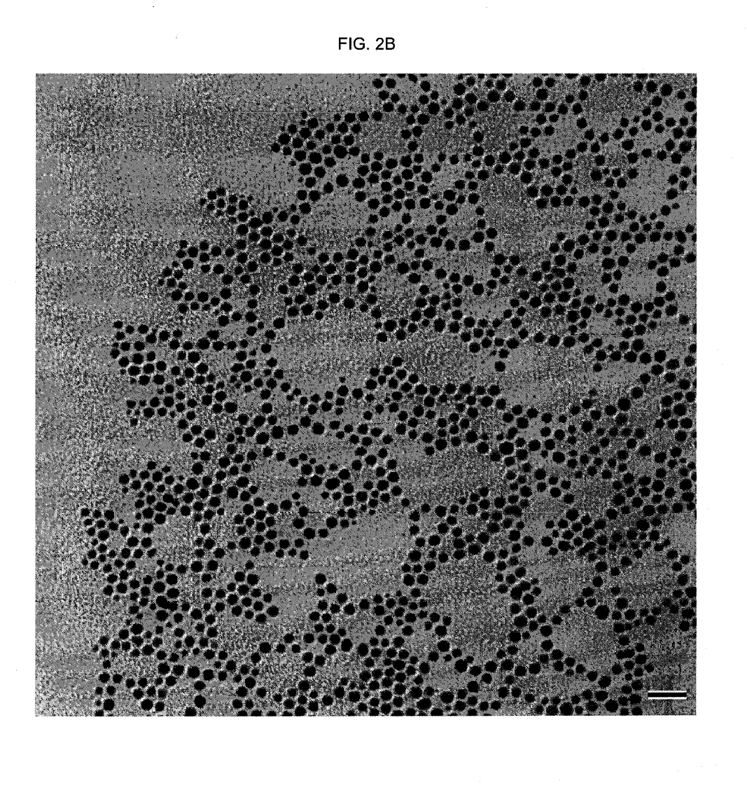

The Ni-NTA-5 nm gold nanoparticles were prepared by the synthesis of 5 nm nitrilotriacetic acid (NTA) gold nanoparticles followed by chelating to Ni²⁺. In addition to Ni²⁺, NTA gold nanoparticles can also chelate to other metal ions such as by way of example only, Cu²⁺ and Zn²⁺. The 5 nm NTA gold nanoparticles were synthesized by adding NaBH₄ (114 mg, 3 mmol) in 5 mL of deionized water to a mixture of HAuCl₄·xH₂O (118 mg, 0.3 mmol), 2-(2′-(2″-methoxyethoxy)ethoxy)ethane thiol (15 mg, 0.08 mmol) and 5-(6′-mercaptohexanoylamino)-1-carboxypentylimino diacetate trisodium salt (32 mg, 0.08 mmol) in 55 mL of 6:1:1 methanol/acetic acid/H₂O with rapid stirring. After two hours of stirring, the 5 nm NTA gold nanoparticles were pelleted down by centrifugation. The crude gold nanoparticles were redissolved in deionized water, and purified by filtration through a membrane with MW cut-off of 30,000. The purified NTA gold nanoparticles were diluted to 1 L with 50 mM MOPs, pH7.9. 600 μL of 0.2 N NiSO₄ solution was added to the NTA gold solution with stirring. After two hours stirring, the solution was concentrated with membrane filtration (MWCO=30,000) and chromatographed over a desalting column, e.g., GH-25. The gold fractions were collected as Ni-NTA-5 nm gold. The prepared Ni-NTA-5 nm gold nanoparticles were nearly monodispersed, and its electron micrograph is shown in FIG. 2.

### Example 2

### Sensitive Detection of a His-Tagged Protein Using Blots.

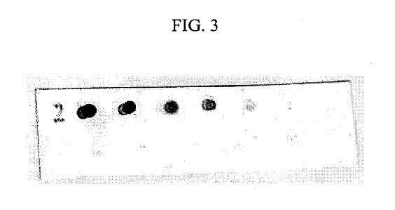

The 6x-His-tagged protein, ATF-1, was spotted onto nitrocellulose, depositing (from left to right) 100 ng, 50 ng, 10 ng, 5 ng, 1 ng, and 0.5 ng per spot. A second row of control proteins (all spots identical), E. Coli extract (1 μL of 2.03 mg/ml total protein, 2.03 μg per spot), was spotted directly below the test target ATF-1 protein spots. The blot was blocked with 5% non-fat dry milk in 20 mM Tris, 0.15 M NaCl, pH7.6 containing 0.1% (w/v) Tween® 20 (TBST), and incubated 30 min. with the Ni-NTA-5 nm gold nanoparticles in 50 mM MOPs pH7.9 (OD_{280 nm}=1.5). The blot was then washed with 10 mM imidazole in TB ST for 2 min. After washing the membrane three times with water, the spots were catalytically enhanced with Gold Enhance EM (Nanoprobes) for 9 min. The membrane was then washed with water and dried. All target spots could be detected (FIG. 3), whereas all control spots were negative, indicating a sensitivity of 0.5 ng.

### Example 3

Labeling of His-Tagged T7 Bacteriophage Viruses with Ni-NTA-5 nm Gold Nanoparticles.

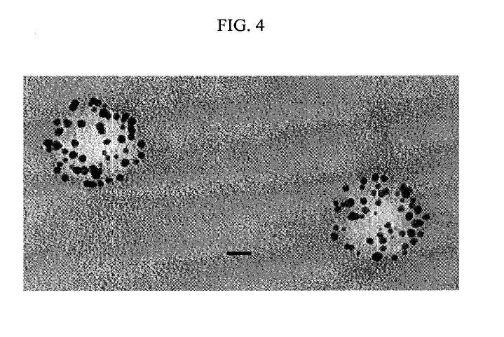

T7 phage was expressed with a His tag insert into some of its coat proteins. The viruses were then incubated with Ni-NTA-5 nm gold nanoparticles and the excess Ni-NTA-5 nm gold nanoparticles were removed by gel filtration on a A5M column. The virus peak was examined by electron microscopy. An example is shown in FIG. 4.

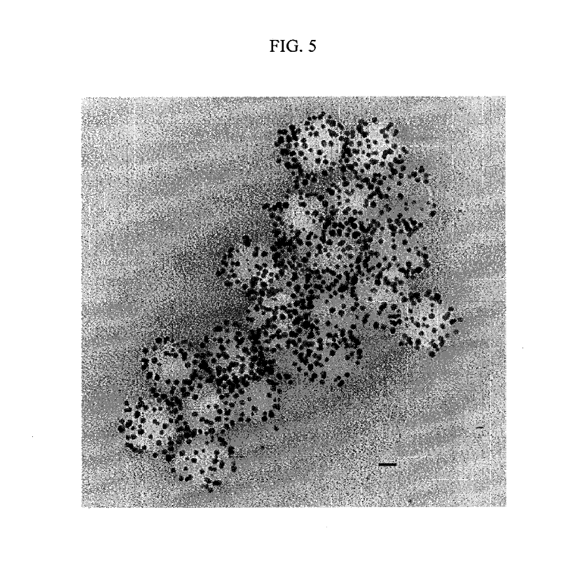

By controlling the reaction and number of linking groups on either the gold nanoparticles or virus, higher order assemblies can be formed, as illustrated in FIG. 5.

### Example 4

### Detecting Polyhistidine Tagged Proteins on Western Blots.

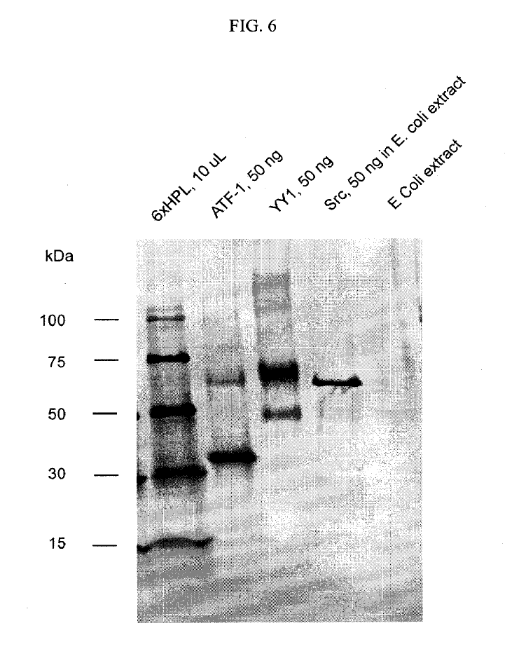

As shown in FIG. 6, the indicated amounts of purified 6x-His-tagged ATF-1 (34 kDa), 6x-His-tagged YY1 (68 kDa), 6x-His-tagged Src (61.7 kDa) mixed with crude extract from E. coli cells, and the crude extract from E. coli cells (1.25 μg total protein per lane) were applied to a 4-15% SDS-polyacrylamide gel. The 6xHis Protein Ladder (6xHPL) consists of five 6x-His-tagged proteins ranging from 15 to 100 kDa. It was loaded as a molecular weight standard, and as a positive control for western blotting. After electrophoresis and Western transfer, 6x-His-tagged proteins were detected by Ni-NTA-5 nm gold nanoparticles followed by GoldEnhance EM (Nanoprobes Inc., catalog #2113). The Ni-NTA-5 nm gold nanoparticles selectively detected all poly His-tagged proteins including 6xHis Protein Ladder, 6x-His-tagged ATF-1, 6x-His-tagged YY1, and 6x-His-tagged Src, but have no detectable binding to the crude extract from E. coli cells.

### Example 5

### Radiotherapy Enhancement Using Ni-NTA-5 nm Gold Nanoparticles.

Gold nanoparticles were found to significantly enhance radiation therapy. They absorb x-rays well and emit electrons that deposit beam energy in their vicinity. Thus, loading tumors with gold nanoparticles can specifically enhance the tumor radiation dose compared to normal tissue. Mice with subcutaneous mouse mammary tumors (EMT-6) are intravenously administered Ni-NTA-5 nm gold nanoparticles. Subsequent irradiation of the tumor region with 30 Gy, 250 kVp photons from a clinical Siemens Stabilipan X-ray generator results in a dramatic shrinkage of the tumors compared to the same treatment without gold.

### Example 6

### Infrared Hyperthermia of Tumors Using Ni-NTA-5 nm Gold Nanoparticles.

Because gold nanoparticles absorb infrared radiation, if they are localized to a tumor, they can be used to heat the tumor specifically upon irradiation. LS 174 human colon cancer cells are subcutaneously implanted in nude mice and tumors develop. These tumors express the carcinoembryonic antigen (CEA). Ni-NTA-5 nm gold nanoparticles are coupled to an anti-CEA antibody and injected intravenously. The gold nanoparticles are found to localize to the tumor. Subsequent irradiation with an infrared lamp with a 665 nm cutoff filter at 2 watts/cm² for a total exposure of 1860 Joules leads to cures.

### Example 7

### Detection of Colon Polyps by Ni-NTA-5 nm Gold Nanoparticles.

Colon tumors are induced in mice by oral administration of the carcinogen azoxymethane. Imaging of the colon after Ni-NTA-5 nm gold nanoparticles injection shows detection of 1 mm tumors. Because the Ni-NTA-5 nm gold nanoparticles are in the vasculature of the tumors, they could be readily distinguished from fecal material that do not increase in radiodensity. A significant practical application of this approach is to distinguish human polyps in the colon by CT without the need for bowel cleansing.

### Example 8

### Kidney and Urinary Tract Radiographic Imaging Using Rapidly Clearing Gold Nanoparticles.

The Ni-NTA-5 nm gold nanoparticles are suspended in phosphate-buffered saline, pH 7.4, and injected intravenously via a tail vein into mice at 1.25 g Au/kg. Mice are then imaged using a clinical CT unit (Philips Brilliance 16) operating at 120 kVp and 146 mA).

### Example 9

### Urinary Tract Imaging Using Ni-NTA-5 nm Gold Nanoparticles

The Ni-NTA-5 nm gold nanoparticles are intravenously injected into mice and x-ray images record. Fine details of the functioning kidney are revealed both in planar and CT x-ray images.

### Example 10

### Tumor Imaging Using Ni-NTA-5 nm Gold Nanoparticles

Tumors have leaky vasculature and gold nanoparticles are found to extravasate specifically there. Ni-NTA-5 nm gold nanoparticles are injected intravenously into mice and tumors and imaged at times thereafter by microCT. Contrasts continue to build over several hours. This class of gold nanoparticles accumulated predominantly around the growing edge of the subcutaneous tumors and enabled positive identification of small, <1 mm thick tumors, which are smaller than those currently clearly identified by x-rays.

1. A composition comprising a plurality of gold nanoparticles bound to at least one multidentate ligand chelated to a metal ion, wherein at least 90% of the plurality of gold nanoparticles have an effective diameter of 5 nm±25%.

2. The composition of claim 1, further comprising His-tagged proteins bound to the metal ion.

3. The composition of claim 1 where the metal ion is Ni.

4. The composition of claim 1, wherein the at least one multidentate ligand is selected from the group consisting of nitrilotriacetic acid (NTA), iminodiacetic acid, tris(carboxymethyl) ethylenediamine, ethylenediaminetetraacetic acid, diethylene triamine pentaacetic acid, and DOTA (1,4,7,10-tetraazacyclododecane-1,4,7,10-tetraacetic acid).

5. The composition of claim 3, wherein the at least one multidentate chelating group is nitrilotriacetic acid (NTA).

6. A composition comprising a plurality of gold nanoparticle conjugates, comprising a plurality of gold nanoparticles bound to at least one multidentate ligand chelated to Nickel (II), wherein at least 90% of the plurality of gold nanoparticles have an effective diameter of 5 nm±25%.

7. The composition of claim 1 or 6, wherein the composition is in a dry powder form.

8. The composition of claim 7 wherein the dry powder form is free of salts, additives or stabilizers.

9. The composition claim 1 or 6, wherein the composition is in a solution form.

10. The composition of claim 9, wherein the composition is in the form of a 0.5 μM in 50 mM MOPS buffer solution.

11. The composition of claim 1, wherein at least 90% of the plurality of gold nanoparticles have an effective diameter of 5 nm±20%.

12. The composition of claim 1, wherein at least 90% of the plurality of gold nanoparticles have an effective diameter of 5 nm±15%.

13. The composition of claim 6, wherein at least 90% of the plurality of gold nanoparticles have an effective diameter of 5 nm±20%.

14. The composition of claim 6, wherein at least 90% of the plurality of gold nanoparticles have an effective diameter of 5 nm±15%.
